(19) United States
(12) Patent Application Publication
(10) Pub. No.: US 2011/0154692 A1
(43) Pub. Date: Jun. 30, 2011

(54) BT' Orthotics, Brilliant Orthotics, BThotics, BThoses

(76) Inventor: Babatunde Olanipekun Adegbile, Dayton, OH (US)

(21) Appl. No.: 12/647,413

(22) Filed: Dec. 25, 2009

(51) Int. Cl. A61F 5/14

(52) U.S. Cl. 36/155

(57) ABSTRACT

The next generation pre-customized/customizable orthotic device “BT' Orthotic Device” or “Brilliant Orthotic Device” otherwise known as “BThotics” or “BThoses” is described that molds around your feet following the natural anatomical gait cycle which can be adjusted manually or automatically. BThotics is a structurally sound orthotic that is so unique and versatile in that it can adjust to your daily needs and preferences be it a mildly high arch, a moderately high arch, a high arch, a narrow foot, a moderately wide foot or wide feet. With BThotics, you do not have to wait 2 weeks for a trial and error test fitting. You would be immediately matched to the appropriate insole/insert/shoe within seconds and better yet, it won't cost you an arm and a leg to get one. For these reasons and many more to be discussed below, BThotics is truly a technological marvel that would revolutionize the orthotic world.

### BACKGROUND OF BTHOTICS

I conceived this idea because I thought that the orthotic industry could use a face lift.

I thought that it would be nice to have a one stop shop orthotic device that would replace several orthotics so that you do not have to keep replacing orthotics or having to buy one for every activity.

The follow scenario may sound familiar: With conventional orthotic devices, you typical need a doctor's order to get a custom made orthotic in-which your feet are cast and sent out to a fabrication center and then you have to wait about 2 weeks before you get to test-fit the orthotic. If you have serious leg problems and/or you are in dire pain from say flat feet syndrome/fallen arches (hyper pronation), posterior tibial tendinitis, ACL, PCL, MCL, arthritis, joint pain, plantar fasciitis (heel pain), chondromalacia, metatarsalgia, shin splints, claw/hammer toes, or bunions just to name a few, now tell me who has 2 weeks to wait for an orthotic? Since these orthotics are custom made, at a going rate of about $400 per pair, they are not cheap neither. Moreover, most orthotic places require you to foot an upfront out of pocket half-down payment since insurance typically would not cover orthotics unless in the rare occasion where it has to do with a disease process such as diabetes for instance. Many a time, you end up wait 2 weeks and only for the orthotic not to fit quite right and after several unfruitful attempts by the “sales rep” of trying to reshape the material to fit your feet, the item ends up getting sent back to be re-fabricated and then you end up waiting even longer and the cycle goes on and on again. At this point, one might as well as save some money by purchasing an OTC (off-the-counter) orthotic for all the trouble. Must I add that often times, custom orthotics are merely overpriced versions of OTC orthotics and often fail to address the issue.

One other issue regarding custom orthotic casts and molds is say for instance you have fallen arches, (hyper pronation), what you need to do is to support your arches, but when a cast of your feet is made you are literally paying for an exact cast of your feet which would probably fit comfortably well, but is it doing anything to support your arch? Casts and molds should be for baseline information only and should be used as a guide because I don't care how much you pay for such a custom piece, if it is not physically supporting your arch such that your fallen arches literally lifts off the floor, and then it is not doing anything to correct the problem. A good orthotic would gradually mold to your feet with time so getting a cast/mold made just for that reason alone is pointless.

When it comes to OTC orthotics, these are typically a lot more economical at an average going rate of about $25 to 30$ and up for a pair. The good thing about these orthotics is that you don't need a doctor's order and you don't have to wait 2 weeks or more to get them. However, have you ever heard of the saying “you get what you pay for”? Well here is a classic example because only after a few weeks of use, expect to pay another visit to the store for yet another pair because from what I have seen, although these may get the job done initially, they tend not to last.

Having given you a breakdown of the two basic kinds of orthotics and their potential drawbacks, I felt the need to innovate something that could revolutionize the orthotic world in a big way. I then put on my thinking hat and after contemplating for a few hours, I pulled out my drawing board and I came up with a rather ingenious idea. That is the “Brilliant Orthotic” device otherwise known as “BT's Orthotic” device which I would be calling “BThoses” or “BThotics” for short.

See . . . I put a lot of thought into this device by attacking the problem firstly as an Engineer (BThotics must be structurally strong and sell itself), secondly as a healthcare professional (would I recommend BThotics?), and thirdly as a potential patient (would I purchase BThotics?) so with BThotics, rest assured that you would be purchasing a uniquely engineered piece of device that would replace all other types of orthotics and save you a boat load of cash and yes, a lot of PAIN in the long run. Hence, because of BThotic's versatility and use-abilities, this makes it stand out from anything else that may be out there which makes it worthy of patent so please read along.

### BRIEF SUMMARY OF BTHOTICS

The next generation pre-customized/customizable “Brilliant Orthotic” device or “BT's Orthotic” device, otherwise known as “BThotics” or “BThoses” is depicted that molds around your feet following the natural-anatomical-gait-cycle (normal kinesthetic posture) which can be adjusted manually or automatically.

BThotic is so unique and versatile in that it can adjust to your daily needs and or preferences be it a mildly high arch, a moderately high arch, a high arch, a narrow foot, a moderately wide foot, and wide feet.

With BThotics, you do not have to wait 2 weeks for a trial and error test fitting. You would be immediately matched to the appropriate insole/insert/shoe within seconds and better yet, it won't cost you an arm and a leg to get one.

For these reasons and many more to be discussed below, BThotic is truly a technological marvel that deserves a patent.

### A BRIEF DESCRIPTION OF THE PHOTOS

I have provided several clear digital images to help depict and illustrate what BThoses look like and how it functions.

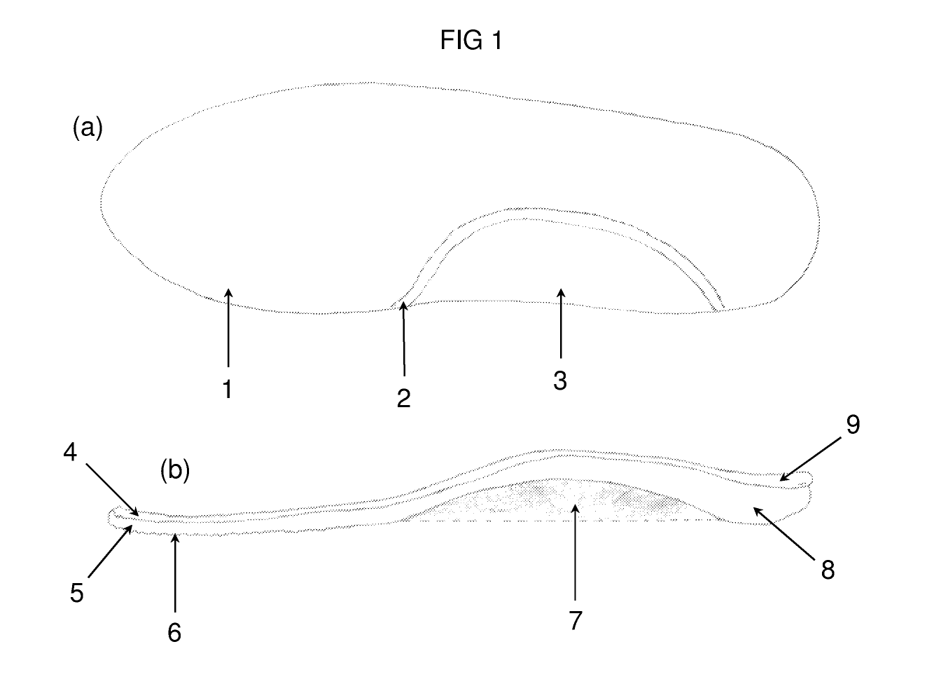

FIG. 1a depicts BThotics Type 1a the right feet from top view.

FIG. 1b depicts BThotics Type 1a the right feet from side view.

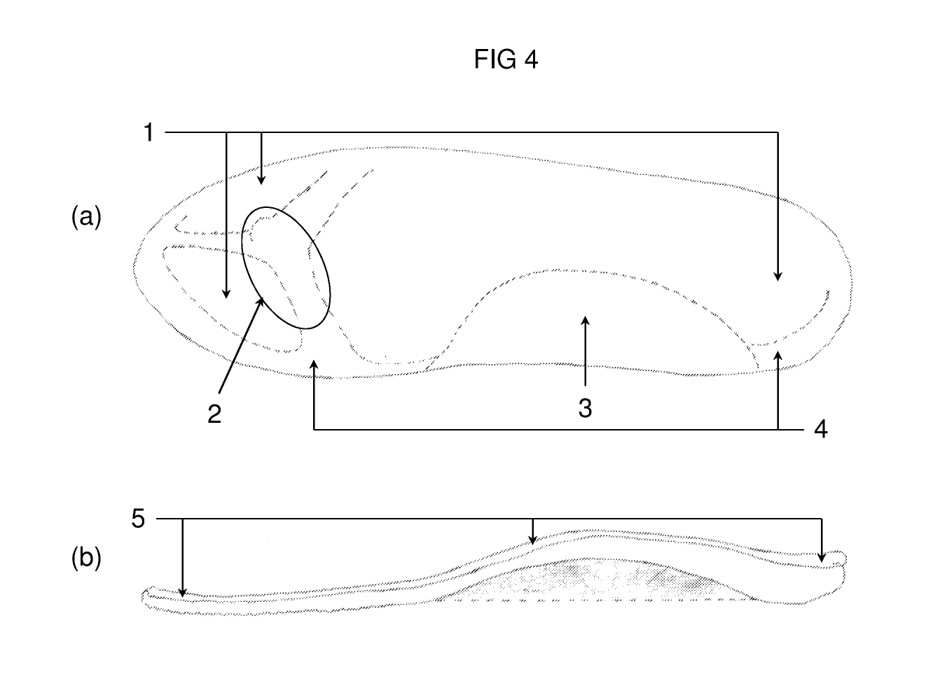

FIG. 1(a) label 1 depicts BThotics Type 1a which is the main body/compartment that composes of the heel/phalangeal single-plate. It also houses FIG. 4a for added comfort, balance, and stability.

FIG. 1(a) label 2 depicts the latching tongue and groove type mechanism of BThotic Type 1a which also doubles as a shock absorber/damper as explained in BThotics Type 2a paragraph 3.

FIG. 1(a) label 3 depicts the one of a kind BThotic swappable customizable arch support.

FIG. 1(b) labels 4 and 9 depict the soft-formed gel-like insole of BThotic from the side view that conforms to your feet to maximize stability and comfort.

FIG. 1(b) labels 5 and 8 depict the main heel/phalangeal sole-plate in which the BThotics arch-plate connects to providing for reinforcement, rigidity, liquid repellence, and anti-microbial while also allowing for ventilation.

FIG. 1(b) label 6 depicts the BThotics anti-slip insole that further provides for rigidity, stability, and balance.

FIG. 1(b) label 7 depicts the swappable/customizable BThotic arch support from the side view that provides stability and arch support as you move through the normal anatomical gate cycle.

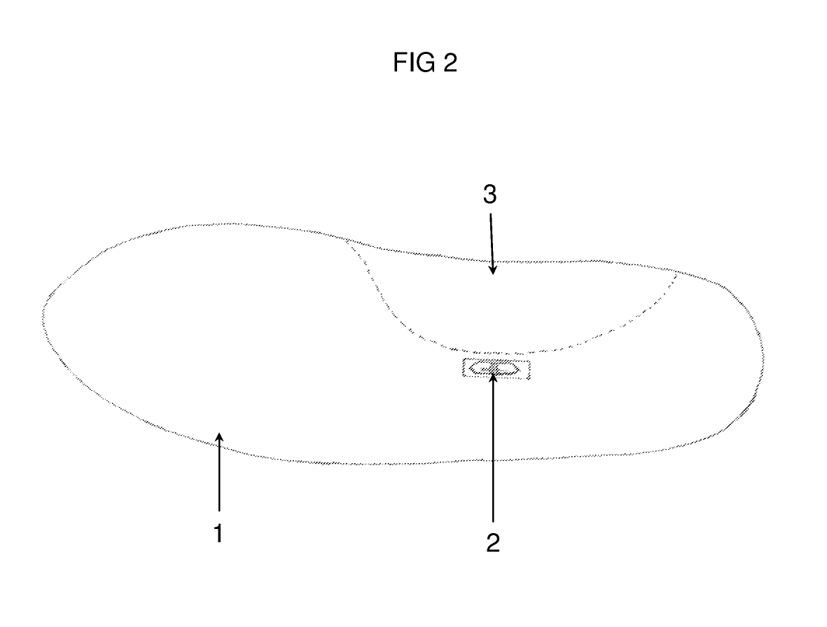

FIG. 2 depicts the right leg of BThotics Type 1 a from bottom-up view.

FIG. 2 label 1 depicts the breathable anti-skid, anti-microbial, moisture-repellant base of BThotics supportive insole from bottom-up view.

FIG. 2 label 2 depicts the innovative one-of-a-kind release/switch/lever/button that separates BThotics from all other competitors out there.

FIG. 2 label 3 depicts the innovative one-of-a-kind swappable arch-piece that separates BThotics from all other competitors out there.

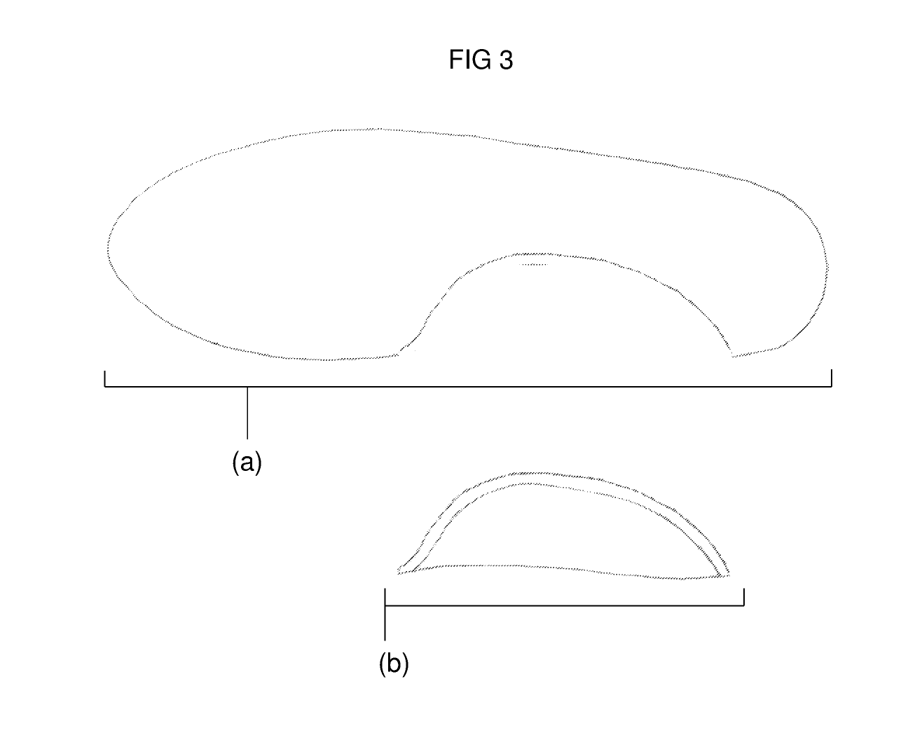

FIG. 3 depicts the right pair of BThotics type 1a from top view.

FIG. 3(a) depicts the main BThotics type 1a insole in-which FIG. 3(b) latches onto for a customizable fit. FIG. 3(a) would also be fitted with a gel-like cushioning material as depicted in FIG. 4a for an optimized comfort.

FIG. 3(b) depicts the innovative detachable/latch-able BThotic arch support piece as being detached which allows for a totally customizable arch support which would be made available in a variety of forms such as, extra soft to rigid. With this unique feature, BThotics is entirely customizable to your taste. Note that this area also serves as a damper/shock absorber which is synonymous to the description given in BThotics type 2a paragraph 3.

FIG. 4 (a) depicts the soft-formed molded insole with optional metatarsal insert which can help to alleviate metatarsal pain.

FIG. 4(a) label 1 as you can see depicts the BThotic insole and how it perfectly molds to your sole imprint creating a depression around your feet for optimal comfort/pain relief.

FIG. 4(a) label 2 depicts an optional metatarsals addition to aide against metatarsal discomfort.

FIG. 4(a) label 3 depicts the innovative swappable/detachable BThotic arch plate.

FIG. 4(a) label 4 depicts the raised part of BThotic insole that molds to and grips around your feet for an enhanced comfort zone.

FIG. 4b depicts in side view, some of the other comfort measures applied to BThotics in hopes of delivering the most comfortable orthotics known to man.

FIG. 4(b) label 5 depicts the raised/griping soft-formed gel-like cushioning from the side view that molds to your feet for added comfort/balance.

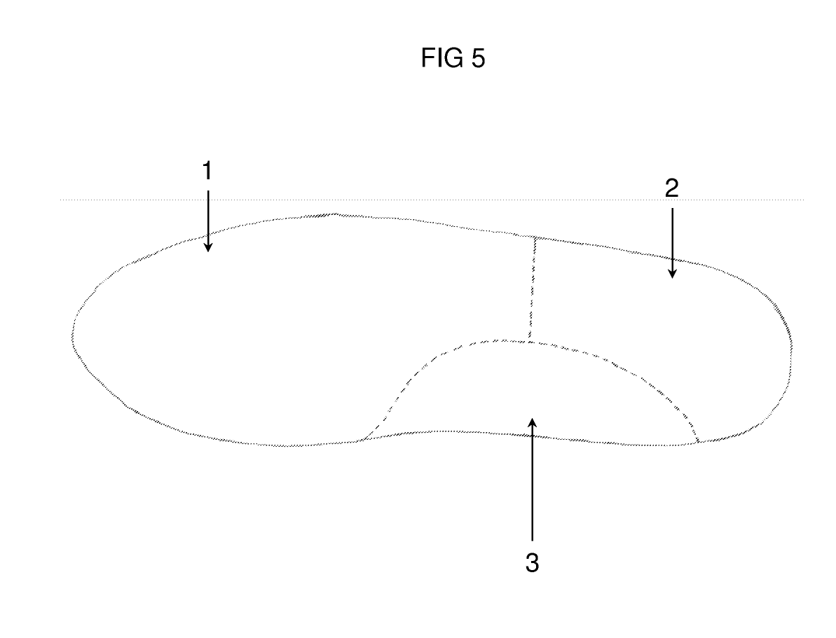

FIG. 5 depicts BThotics type 1b which takes BThotics type 1a a step further by not just allowing for a customizable arch support, but also allowing for customizable heel and phalangeal plates. These innovative concepts set BThotics apart from all other competitors.

FIG. 5 label 1 depicts the fully customizable/swappable phalangeal plate for a perfect fit; these phalangeal plates also come in optional metatarsal version as depicted in FIG. 4(2).

FIG. 5 label 2 depicts the swappable/customizable heel plate which allows for the selection of just the right cup/bust size for the heel.

FIG. 5 label 3 depicts the quick detachable arch plate via a tongue and grove type latching system or the like.

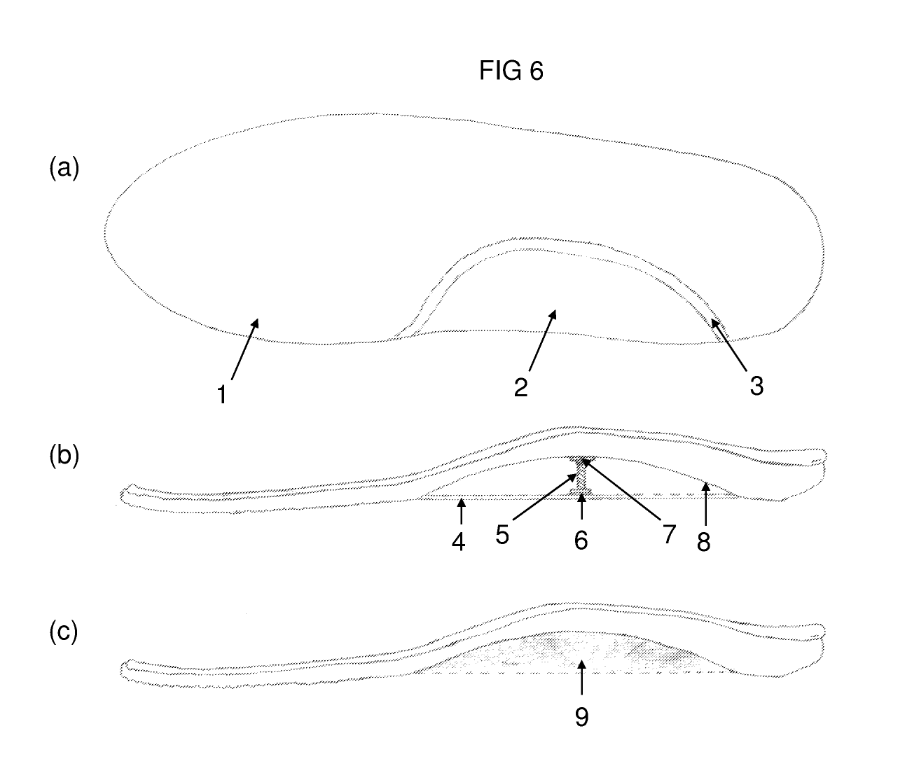

FIG. 6 depicts BThotics type 2 which is available in manual mode and in a self adjusting/programmable automatic mode.

FIG. 6a depicts BThotics type 2 which is equipped with a mechanical actuating device which serves to adjust your arch (ex: mild, semi-rigid, rigid) as you please.

FIG. 6a label 1 depicts the main base/body of BThotics type 2 which is fitted with a comfortable insole as depicted in FIG. 4.

FIG. 6a label 2 depicts the BThotics arch-plate that is tittered to the main insole and is slightly actuate-able from say 1 cm to 1.5 cm with “0 cm” being neutral arch height. This allows for a seamlessly customizable arch height.

FIG. 6a label 3 depicts the connecting portion of BThotics type 2 that connects the arch to the main insole/body. It is to be made up of flexible but tough material that would expand/adjust to the arch height while still providing support. Unlike the quick release latch-able tongue and groove system depicted in BThotics type 1a label 3, here we have a variable arch height adjustment system that also acts as a shock absorber/damper—ingenious.

FIG. 6b depicts BThotics type 2 with a transparent protective covering for labels 4 through 8 such that one can see and appreciate its inner workings which also brings about a rather cool look.

FIG. 6b label 4 depicts the base-plate that helps to reinforce and stabilize the Bthotic arch.

FIG. 6b labels 5, 6, and 7 collectively depict the mechanical actuating shock absorbing/damping system that serves to adjust the arch height with a 2.5 cm range from say for example −1 cm to 1.5 cm with “0” (1 cm height) being the neutral arch height. Also, FIG. 6 labels 5, 6, and 7 collectively serve to prevent lateral (outward rotation) and medial (inward rotation) tilting by slightly counter adjusting hence acting as a shoe wedge/cork.

FIG. 6b label 5 depicts the shaft of the mechanical actuating shock absorbing damping system which directly aides in arch height adjustment.

FIG. 6b label 6 depicts the base of the actuator that directly connects to the base-plate FIG. 6b label 4 and also acts as arch support hereby stabilizing the arch.

FIG. 6b label 7 depicts the top part of the actuator/solenoid that attaches to the bottom part of BThotic arch FIG. 6b label 8.

FIG. 6b label 8 depicts the outer BThotic arch shell which directly connects to the actuating shock absorbing/damping system allowing for a seamless and adjustable BThotic arch for a one size fits all.

FIG. 6c label 9 depicts a solid version of the protective covering/guard to FIG. 6b labels 4 through 8 such that at a glance BThotics type 2 appears as though it were the ordinary/average orthotic.

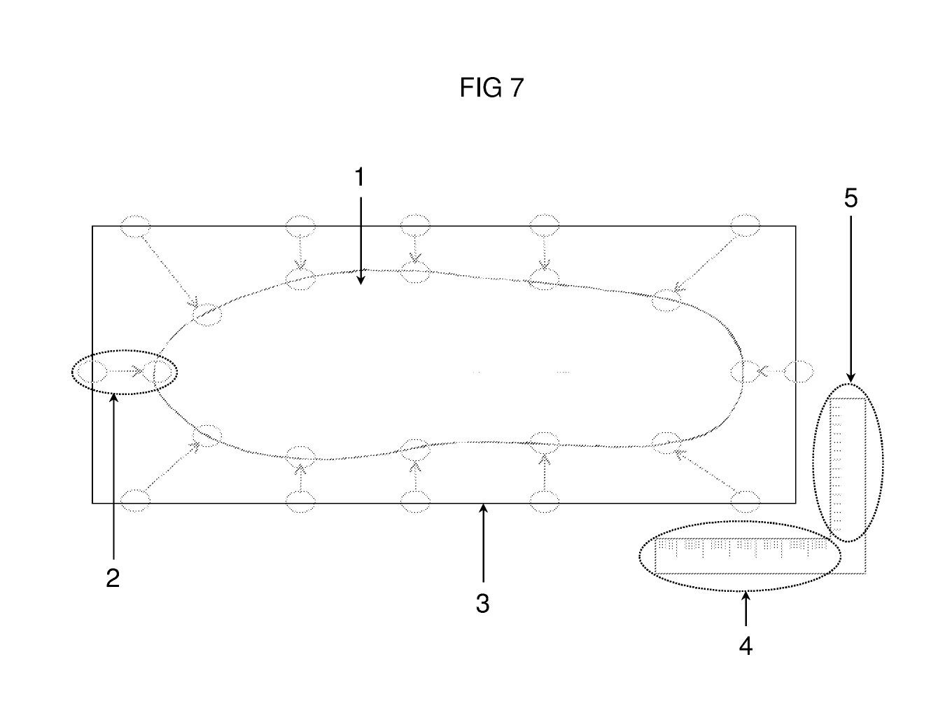

FIG. 7 depicts a generic BThotic selecting device for illustration purposes which would be used to explain BThotic selector types 1a, 1b, 2a, and 2b.

FIG. 7 label 1 depicts a sample representation of a mold/cast of a person's feet for illustration purposes.

FIG. 7 label 2 depicts how label 3 (a cast/mold making substance) molds/wraps around your feet to form a perfect representation of your feet as exemplified by the sample foot imprint as depicted in FIG. 7 label 1.

FIG. 7 label 3 depicts a clear see-through glass/plastic type compartment/molding that fits like a shoe and adjusts to variable sizes as needed for a perfect casting.

FIG. 7 labels 4 and 5 depict a scaling/measuring device that is comparable to a clear/see-through artistic ruler with measuring grids/markings superimposed onto label 3. With these markings, BThotics can immediately and accurately match a foot to the best possible BThotics. You can read more about this in the detailed section.

### DETAILED DESCRIPTION OF BTHOTICS

Brilliant Orthotics/BT's Orthotics/BThotics/BThoses is a one-of-a-kind next generation orthotic device that is engineered to be a single adjustable/customizable orthotic which would save you boat loads of cash in the long run. Unlike any other orthotic device out there, BThotics has foolproof solutions so you can consider BThotics to be the epitome of all other orthotics because this single orthotic replaces having to purchase dozens of orthotics.

A few known orthotic issues are: a weakened/fallen arch support of the orthotic over time, insufficient arch support from the orthotic to start with, inappropriately fitted orthotics, and a single size non customizable orthotic.

Bthotics serves to address the aforesaid issues by introducing foolproof state of the art, solutions to these problems which would be further discussed below.

### Types of BThotics

There are roughly 3 basic types of BThotics.
1.) Type 1 is a one-of-a-kind innovative next generation orthotic device that essentially allows you to physically adjust its shape and or size. To further illustrate how this is possible, BThotics Type 1 is further subdivided into 2 categories.

A. BThotics-Type-1a:
With this Version of BThotics, for the very first time, you can now physically adjust arch sizes/heights as you please. This is because BThotics-Type-1a allows you to swap out the arch as depicted in FIG. 3 so that you have the liberty of going from a mildly rigid arch to a semi rigid/rigid arch as you please depending on your taste or activities you engage in.
So you can think of BThotics-Type-1a as a quick release latch-able/tongue and groove/rabbit and dado orthotic device that allows you to conveniently slide the arch in onto the main orthotic. Isn't that great? This way, you have full control over how soft, hard or rigid you want your arch to be so that you don't have to keep shopping around yet again for the same-old orthotics costing you a fortune and a lot of unnecessary physical PAIN in the long run.
Envision a single orthotic device BThotics that comes with several replacement/swappable arches so that you have one arch per activity? Say for instance you work long hours while standing on your feet all day, you might want a more rigid/supportive swappable arch, or say you want something more soft/comfortable, then you might want a flex-able swappable arch, and lastly, say you want something in-between, then you might want a semi-rigid swappable arch.
Another scenario is when you are new to orthotics for instance, you may want to start out with a more comfy/flexible BThotic arch, and then as time goes on and you begin to get used to the natural anatomical gait cycle, then you may want to swap in a semi-rigid BThotic arch and finally a more rigid/supportive BThotic arch.
With this innovative approach you can now buy a single heavy duty long lasting orthotic device and all you would need to do is to swap the arches as you please so for instance if you have any problem whatsoever with an arch-piece, you can simply swap in a replacement arch-piece. In addition to that, even if you would need to purchase replacement arch-pieces, that would be a lot cheaper than having to buy the entire orthotic over again. However, rest assured because that won't be the case as BThothics would be built with the most durable and yet cost effective materials out there and when I say durable I mean business because I want BThotics to last a life time.

B. BThotics-Type-1b:
BThotics-Type-1b is similar to BThotics-Type-1a, but it takes it a step further. With BThotics-Type-1b you are not just given control over your arch, but for the very first time, you can now fully customize and control the entire BThotic.
In achieving this, I basically partitioned BThotics-Type-1b into 3 parts as any basic human foot/sole can simply be divided into 3 parts that is to say your toes and or phalanges, your heel, and of course, your arch/elevation.
Similarly to BThotics-Type-1a, BThotics-Type-1b would have a quick release latch-able/tongue and groove system(s) that would allow for a quick and easy swap of the arch plate, the heel plate, and the toe/phalange plate, independently of each other.

Arch Plate:
BThotics-Type-1b customizable arch plate is synonymous to the aforesaid BThotics-Type-1a customizable arch insert which allows for you to not only set the height of your arch, but also allows the freedom to choose how rigid you want your arch support.

Heel Plate:
The customizable heel plate is yet another innovative approach to revolutionizing the orthotic world. This added option allows for you to be able to closely and accurately match up heel cup sizes such that BThotics literally molds around your heel. For example, a person with a more defined heel would be matched with a deeper heel cup which would serve to align and stabilize the body as it flows through the normal anatomical gait cycle. There would be several heel cups to choose from so that you can be closely matched with the most appropriate heel cup.

Toe/Phalangeal Plate:
When it comes to the phalangeal plate, since width sizes vary from person to person, BThotics-Type-1b allows you to pick from various width sizes such as narrow, standard, wide, and extra-wide just to name a few. This allows for you to customize your personal BThotics to suit your feet rather than being limited to just one standardized size that may or may not fit you right. Take for instance, everyone could not possibly be fitted to a size 13 shoe right? And that is exactly why we have various shoe sizes so that you can be matched to the most appropriate shoe for maximum comfort and functionality.
One more thing:
I would like to point out that unlike most of all other orthotics out there that tend to incorporate metatarsals toe inserts/molding within the insole at a predetermined universal location, which may or may not align/fit to every foot, BThotics on the other hand allows for the metatarsals to be placed right where you need it based on your feet rather than it being placed in some randomly predetermined position as no two feet is exactly the same size, shape, or width.
2.) I am very much excited to unveil BThotics Type 2 as it takes BThotics Type 1 to a whole new level entirely. Now unlike BThotics Type 1 that allows you to physically adjust its shape and or size by swapping arches, BThotics Type 2 does essentially the same thing, but without you having to swap arches. I don't know if you are starting to get the picture already but if not, I would further illustrate how this is possible in the following paragraphs.
   Unlike BThotics Type 1a for instance, that has 2 separated parts (the swap-able/detachable arch and the main insole); BThotics Type 2's arch is always tittered to the main insole via a mechanical actuating shock absorbing damper that serves to adjust the arch height. For example a maximum arch height of 2.5 cms would adjust from −1 cm to +1.5 cms with “0” (or 1 cm) being neutral arch height. Isn't that wonderful? With such customizability, a person new to orthotics could start out at say 1 cm and slowly walk their way to a more natural arch height say about 2 cms or more. This is what makes BThotics stand out from every other orthotic device out there because unlike most orthotic insoles/inserts you may find that only after using it for a few months that the arch is fallen and then you have to either replace it or pay for a costly customizable that would eventually succumb to the same faith.
   I would now like to disclose the two basic types of BThotics-Type-2.

BThotics-Type-2-M:
With this kind of BThotics, you can adjust its height by manually cranking or un-cranking a lever which would be conveniently located on the insert. This lever in turn actuates the shock absorber/damper which then controls arch height. There is to be an easy to read meter/gauge that would indicate how high the arch is set at all times.

BThotics-Type-2-A:
This is the automated version of BThotics that allows for you to conveniently control arch height at the click of a button. Since this BThotics is automated, it would probably require some kind of power so a few preferably rechargeable wrist-watch type mini/portable batteries or some other form of alternate energy source should do the trick. This type of BThotics is also better suited with digital readouts that would display arch height and other appropriate data.
Other functions include memory/recall and being able to preprogram/set specific heights for specific activities. So for instance, if you would be standing for long periods of time at your job, you would probably want the arch to be more rigid/high and BThotics allows you to do this with a simple click of a button.
One more thing:
When describing BThotics Type 2, I kept stressing the phrase “actuating shock absorber/damper” and this is because I am not just talking about a basic actuating device here. Take for example, if you find your comfort zone to be at height 2.25 cm, BThotics' special actuating shock absorber/damper would slightly depress to say for instance 2 cm in other to absorb shock as needed and then go back to the preset height. This way, BThotics stays rigid while still absorbing shock as you go through the normal gait cycle. This actuating/shock option is such a unique function to BThotics that it transcends all other orthotics out there. I will now move on to BThotics Type 3.
3.) BThotics Type 3:
   BThotics Type 3 combines BThotics Types 1 and 2 and puts all that customizability and functionality into a shoe. So BThotics Type 3 is basically an orthotic shoe or a BThotic shoe if you will.
   BThotics Type 3 comes in a variety of shapes, types, and sizes and it allows for you to pick anywhere from gym shoes to dress shoes and so on. These BThotics will be designed to look great so you don't have to worry about having to wear heavy, ugly, bulky orthotic shoes around. Simply bring in your favorite shoe, and you shouldn't have a problem finding a closely matching BThotics otherwise, one can be customized for you.

### Material

I would like for BThotics to last a life time, and in other to achieve this, the structural frame work has to be sound. Hence, the following paragraphs explain some of the materials in-which this pre-customizable/customizable innovative orthotic arch supportive device would be made up of.

First and foremost, BThotics has to be tough enough to withstand years of repeated abuse from machine washing.

### Insole (Top/Inside):

The entire BThotics especially the insole would be made with any of the various anti-microbial products that would help to cut down on odor and the potential for foot infections. Also, the insole and the entire BThotics would be ventilated and breathable which serves as a means to further help cut down on germs and odor.

The insole would have a deep heel cup that would be matched to your heel/bust which would provide for balance, stability, and proper body alignment as you move through the normal anatomical gait cycle.

The insole would also consist of a well formed soft and comfortable top layer that would help facilitate extra comfort to create a soothing experience.

Note that the great thing about BThotics is that it comes in so many lengths, widths, and sizes that it would not be necessary to have to trim it. You simply move on to the next length/width/size. However, BThotics is trim-able.

### Bottom of Sole:

At least a few criteria have to be met when it comes to the bottom sole. That is to say that it has to be tough and rigid, provide extra added stability (while retaining its shape) and it must be anti-slip. The sole is especially important when it comes to medial rotation (supination) and lateral rotations (pronation).

### Framing:

It is always a good idea for anything that is meant to carry weight to be structurally sound otherwise, it would eventually fail and that is exactly want I am trying to prevent. Take your house for instance does it have framing? How about your car? Or better yet, how about the human anatomical framing system (otherwise known as the skeletal system)? Since I want BThotics to last a life time, a sound structural framing is necessary. See, this is exactly what makes BThotics stand out from the crowd.

To be structurally sound, BThotics could be reinforced with anything from metal, steel, carbon fiber, fiber glass, and tungsten carbide just to name a few examples. I would also like to mention that the different parts and components of BThotics are to be glued, fused, and/or triple-stitched together when necessary to ensure for a strong and long lasting virtually inseparable bond.

Unlike most other orthotics, this framing is the key that helps to hold BThotics together so that it maintains its original solid shape at all times. Once the frame is built, other softer products like plastics, rubber, and gel could be used to fill in around the frame so as to create an extremely soft, but yet rigid and comfortable orthotic device that would simply trump anything else out there.

### The Instant BThotic Selection and Matching System

The instant BThotic selection system is a system that allows for a quick orthotic selection to be made without having to randomly test-fit a bunch of BThotics or worse yet have to wait 2 weeks for a custom orthotic. Now, this system works a little different from previous conventional casting/molding. Although I could easily dream up a dozen ways of doing this, I narrowed it down to two basic types of selectors.

### BThotic Selector Type 1 (BTST1):

This is the basic version of the selector that would aid in picking out the right BThotic for your feet.

Now, BTST1 allows for your feet to be matched to a precustomized/customizable BThotic device within seconds. This is very handy because rest assured that you would be getting the best match to your feet and besides, you won't have to wait 2 weeks to test fit.

BTST1 is to be made of preferably a clear see through glass/plastic compartment that would fit you like a shoe and it is adjustable to variable sizes so don't worry as there is a size for everyone. BTST1 would then contain a cast/mold material that would copy the exact image of your feet. Now since BTST1 is made of clear material, it is easy to see calibrated lines and/or markings as the mold appears to be superimposed over BTST1 such that important information like arch height, foot length, and foot width can easily be retained. Think of BTST1 as being a clear/see-through artistic ruler that allows the artist to superimpose itself onto his drawings for precise placement of lines. Or better yet, think of BTST1 as a multi CDR spindle plastic see-through cover, except that BTST1 would fit you like a shoe and it has a mold/cast material inside of it. Now also think of this CDR spindle cover having calibrated markings on it. These markings then allows for BTST1 to immediately and accurately match your feet to the best possible BThotics. Please see FIG. 7 and its descriptions for details.

### BThotic Selector Type 2 (BTST2A and BTST2B):

When it comes to BTST2 there are two subcategories. One is comparable to a snapshot device while the other is more of a scanning device. Both imaging devices would accurately match a person's foot to the best possible BThotics by compiling exact 2d and/or 3d images all within seconds.

### BTST2A:

Unlike BTST1, this version of BTST2 is different in that it relies on snapshot images to make selection. BTST2A would obtain images of a mold imprint from different angles say side views, top view/bottom views, rear/front views and then send the 2d images to a processor that would then compile the information into a perfect 3d representation of your feet which would then be used to select matching BThotics.

### BTST2B:

BTST2B is different from the BTST2A in that it actually scans the cast/mold imprint created and then immediately sends the information to a processor that would then convert it to 2d or 3d. One of two ways that BTST2B would scan the mold imprint is via a sensory device that would sense for solid objects/surfaces. A second way is for BTST2B to be equipped with a manual scanning device that would physically contact and traverse the mold imprint of your feet and then output an exact 3d representation that would be matched to the perfect BThotics.

### Bells and Whistles

Firstly, as an added option, BThotics can be rigged up with tiny feet massagers so that you can get a soothing massage as you go through the anatomical-gait-cycle (walk).

A second optional feature I would like to throw in is that BThotics can help soothe your sole via its heat/cool or warm/cold compresses option which would help to loosen up tension in your feet as you go through the normal gait cycle.

These are just a few extra options of BThotics that would be made available to consumers upon request.

In no way are these memoir and/or BThoses/BThotics being limited to the above said descriptions and/or photos. These are merely suggestions as to what BThotics/BThoses would look like and how it would function. I, Babatunde Adegbile reserve the rights to change, update, combine, or modify any of the various portion(s) of this memoir and/or BThotics/BThoses at a later date if deemed necessary.

### The Feasibility of BThotics

As you can see, I (BT) have put in a lot of time, effort, and dedication to show that BThotics, a one of a kind innovative idea is truly feasible as illustrated in the attached drawings. The only thing now left to be done is for my fellow engineers (manufacturers) to actually make BThotics so that it can be put to walk—BT smirks. I mean, BThotics is not just another flimsy orthotic, but rather it is a unique piece of ingenuity that one could consider to be man's sole best friend—BT smirks again. Thank you very much for your time.

What is being claimed is:

1. The next generation pre-customized/customizable orthotic device “BT' Orthotic Device” or “Brilliant Orthotic Device” otherwise known as “BThotics” or “BThoses” which is a cost effective one stop shop orthotic device, said orthotic device having a quick selection device, said orthotic device having five basic types/versions BThotics type 1a, BThotics type 1b, BThotics type 2M, BThotics type 2A, and BThotics type 3, said orthotic device being adjustable manually and/or automatically to suit a specific purpose and/or activity.

2. BThotics type 1a of claim 1, wherein said orthotic supportive arch-plate as depicted in FIG. 1(a) label 3 is swappable/detachable from the main orthotic insole as depicted in FIGS. 3a and 3b which serves as a means of customizing and controlling the arch height and pressure while maximizing comfort for the ultimate customizable orthotic as explained in the detail section.

3. BThotics type 1b of claim 1, wherein said orthotic device does not only have a customizable/swappable arch support system, but also allows for the customizing of said heel-plate and said phalageal-plate as depicted in FIG. 5 and as explained in the aforesaid detail section.

4. BThotics type 2M of claim 1, wherein said orthotic device has a mechanical strut that serves to not only adjust arch height/tension, but also acts as a shock absorber/damper which can be manually controlled to suit a specific purpose or activity.

5. BThotics type 2A of claim 1, wherein said orthotic device has an automatic but yet, a user-friendly override-able mechanical strut that serves to not only adjust arch height/tension, but also acts as a shock absorber/damper which automatically suggests arch height/tension based on the activities you engage in, all the while preventing medial and lateral rotation/tilting as you move through the normal anatomical gait cycle.

6. BThotics of claim 1, wherein said custom/pre-customizable orthotic shoe or BThotics Type 3 combines technology and functionality from above-said BThotics Type 1 and Type 2 and puts it all into a designer orthotic shoe, said orthotic shoe having being equip-able with not limited to metatarsal insert(s), but also equipped with optional in-built features such as, tiny foot massagers and cold/hot compresses for the ultimate foot relief.

7. The BThotic quick selection device of claim 1, wherein said orthotic selector device serves as a means of quickly, effectively, and instantly matching a BThotic to an arch seamlessly, thus eliminating the unnecessary need for having to wait for custom orthotics.

8. BThotics of claim 2, wherein said detachable customizable arch-plate support houses a latching tongue and groove type mechanism as depicted in FIG. 1(a) label 2 that also doubles as a shock absorber/damper as you move through the normal anatomical gait cycle as further explained in the detail section under BThotics Type 2a paragraph 3.

9. BThotics of claims 2, 3, and 4, wherein said orthotic arch-plates are solid just like shoe cork or the like such that medial and lateral tilting/rotation and/or fallen arches are controlled/prevented hence promoting an anatomically normal gait.

10. BThotics of claim 1, wherein said main orthotic insole as depicted in FIG. 2 label 1 houses a release/switch/lever/button as depicted in FIG. 2 label 2 that latches and unlatches the detachable customizable arch-plate as depicted in FIG. 2 label 3.

11. BThotics of claims 2, 3, 4, and 5, wherein said soft-formed molded insole as depicted in FIG. 4a comprises of phalangeal-plate that is essentially equip-able with not limited to metatarsal insert(s), but also equip-able with optional in-built features such as, tiny foot massagers and cold/hot compresses for the ultimate foot relief.

12. The BThotic quick selection device of claim 6, wherein said orthotic selector device is not only fitted with special casting/molding material that would copy the exact image of your feet, but it is also made up of translucent material such that it is easy to see calibrated lines and/or markings superimposed over the molding allowing for important information such as arch height, foot length, and foot width to easily be retained which would be instantly matched to the perfect BThotics.

13. The BThotic quick selection device of claim 6, wherein said orthotic selection device obtains snapshot images of a mold imprint from different angles such as side view, top view/bottom view, rear/front view and then sends the 2D images to a processor that would then compile the information into a perfect 3D representation of your feet which would then be used to instantly select the perfectly matching BThotics.

14. The BThotic quick selection device of claim 6, wherein said orthotic selection device scans the created cast/mold imprint via a sensory device that would sense for solid objects/surfaces and/or a manual scanning device such as tiny rollers and/or markers that would physically contact and traverse the mold imprint and immediately port the information to a processor that would then convert it to an exact 2D or 3D representation that would instantly be matched to the perfect BThotics.
